(19) United States
(12) Patent Application Publication
(10) Pub. No.: US 2004/0242972 A1
(43) Pub. Date: Dec. 2, 2004

(54) Method, system and computer product for prognosis of a medical disorder

(75) Inventors: Sudeshna Adak, Bangalore (IN); William Phillip Gorman, Niskayuna, NY (US); Kati Illouz, Schenectady, NY (US)

Correspondence Address: General Electric Company, CRD Patent Docket Rm 4A59, P.O. Box 8, Bldg. K-1, Schenectady, NY, 12301, US

(73) Assignee: General Electric Company

(21) Appl. No.: 10/446,494

(22) Filed: May 28, 2003

(51) Int. Cl. A61B 5/00

(52) U.S. Cl. 600/300; 128/920

(57) ABSTRACT

Method, system and computer product for prognosis of a medical disorder. In one embodiment, a request to provide prognosis decision support for the patient is received. Medical data relevant to the patient such as longitudinal medical data is extracted. Predictive modeling techniques relevant to the patient from the longitudinal medical data are derived. The predictive modeling techniques are then used to predict a clinical outcome for the patient from the medical data. The predictive modeling techniques are formulated using data mining techniques that are capable of detecting correlations in repeated measurements associated with the longitudinal medical data. The predictive modeling techniques then utilize the correlations for determining the medical prognosis for the patient.

### BACKGROUND OF THE INVENTION

The invention generally relates to a medical decision support system and more specifically for the prognosis of a medical disorder.

Medical decision support systems typically use patient data as a guide to clinical decision-making. In addition, medical decision support systems store information on a very large number of socio-demographic variables, physical and medical history, a variety of biochemical laboratory tests, genetic information, radiological images and scans, environmental risk factors, etc in medical databases. The medical databases usually comprise longitudinal or temporal medical data, which is an important special class of patient data, in which multiple measurements are made on the same patient over time. Longitudinal medical data typically comprises multiple records from a single patient collected in several clinic visits spread over days or months or years. Longitudinal databases have been used in medical studies that include Alzheimer's Disease (AD), Huntington's Disease (HD), other neurodegenerative diseases, cardiovascular diseases, AIDS, cancers and other chronic diseases.

Existing medical decision support systems for medical diagnosis and prognosis use data modeling techniques that automatically select prognostic features for optimal prediction of clinical outcomes. Clinical outcomes indicate a variety of outcomes in a patient's condition such as disease status, disease severity, disease progression, risk of hospitalization, risk of mortality, and time to events such as survival or hospitalization. Prognostic features are characteristics of patients that affect future clinical outcomes for the patient. Prognostic features are disease specific. Some examples include age, brain volumes in cases of Alzheimer's disease, CD4 cell counts in cases of AIDS and TNM stage in cases of cancer.

Some of the commonly used data modeling techniques in medical decision support systems include classification and regression tree algorithms and neural networks. These data modeling techniques are not specialized to utilize the additional power of longitudinal medical data for improved clinical decision making because these techniques take into account only single observations for each patient. Consequently, the ability of these existing types of medical decision support systems to improve the clinical decision making process and aid in clinical research is affected since they employ data modeling techniques that do not make use of time-indexed data to predict a future course of the clinical outcome over time.

Therefore there is a need for the creation of medical decision support systems that employ data modeling techniques that are capable of utilizing longitudinal or temporal data from medical databases and the patient's medical history to determine a prognosis for a patient.

### BRIEF SUMMARY OF THE INVENTION

In one embodiment, a method and a computer readable medium to determine a medical prognosis of a patient with a medical disorder is provided. In this embodiment, a request to provide prognosis decision support for the patient is received. A plurality of medical data relevant to the patient is extracted. The plurality of medical data comprises a plurality of longitudinal medical data. A plurality of predictive modeling techniques are then used to predict at least one clinical outcome for the patient from the plurality of medical data. The plurality of predictive modeling techniques are formulated using a plurality of data mining techniques that are capable of detecting correlations in repeated measurements associated with the plurality of longitudinal medical data. The plurality of predictive modeling techniques utilize the correlations for determining the medical prognosis for the patient.

In a second embodiment, there is medical decision support system for prognosis of a medical disorder. The system comprises a data storage component configured to store a plurality of medical patient data comprising longitudinal medical data. The system further comprises a prediction engine component coupled to the data storage component. The prediction engine component comprises a plurality of predictive modeling tools that predict at least one clinical outcome for a patient. The plurality of predictive modeling tools are formulated using a plurality of data mining tools that are capable of detecting correlations in repeated measurements associated with the plurality of longitudinal medical data. The plurality of predictive modeling tools utilize the correlations for determining the medical prognosis for the patient.

In a third embodiment, there is a method and a computer readable medium to determine a medical prognosis of a patient with a medical disorder. In this embodiment, a plurality of medical data relevant to the patient is extracted. The plurality of medical data comprises a plurality of longitudinal medical data. A plurality of predictive modeling techniques are then used to predict at least one clinical outcome for the patient from the plurality of medical data. The plurality of predictive modeling techniques are formulated using a plurality of data mining techniques that are capable of detecting correlations in repeated measurements associated with the plurality of longitudinal medical data. The plurality of predictive modeling techniques utilize the correlations for determining the medical prognosis for the patient.

### BRIEF DESCRIPTION OF THE DRAWINGS

FIG. 1 shows a schematic of a general-purpose computer system in which a medical decision support system for prognosis of a medical disorder operates;

FIG. 2 shows a top-level component architecture diagram of the medical decision support system that operates on the computer system shown in FIG. 1;

FIG. 3 is a high level flowchart describing the steps performed by a predictive models subcomponent of a prediction engine component of the medical decision support system shown in FIG. 2;

FIG. 4 is a flowchart describing a step of FIG. 3 in further detail;

FIG. 5 is a flowchart describing a data mining technique for longitudinal medical data based on regression trees;

FIG. 6 is a flowchart describing a step of FIG. 5 in further detail;

FIG. 7 shows a structure of a regression tree growing with two prognostic features X₁ and X₂;

FIG. 8 shows a partition of the feature space corresponding to the splits defined by the regression tree shown in FIG. 7;

FIG. 9 is an illustration of a data mining technique for longitudinal medical data based on neural networks;

FIG. 10 is a flowchart describing a data mining technique for longitudinal medical data based on neural networks;

FIG. 11 describes a step of FIG. 10 in further detail;

FIG. 12 is a flowchart describing the steps performed by a monitoring and validation component of the medical decision support system shown in FIG. 2;

FIG. 13 is a flowchart describing the steps performed by a similarity search subcomponent of a prediction engine component of the medical decision support system shown in FIG. 2; and

FIG. 14 shows a screen display that the prognosis display subcomponent may present to a user of the medical decision support system.

### DETAILED DESCRIPTION OF THE INVENTION

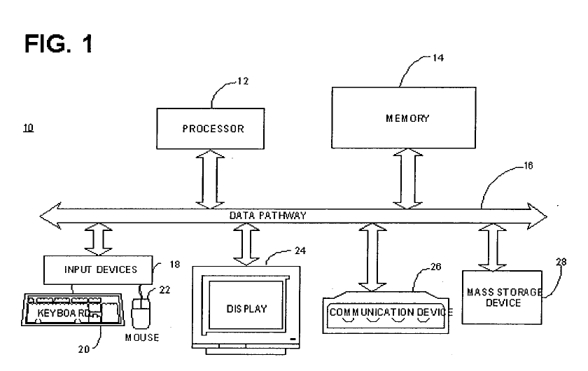

FIG. 1 shows a schematic of one embodiment of a general-purpose computer system 10 in which a medical decision support system for prognosis of a medical disorder operates. The computer system 10 generally comprises at least one processor 12, a memory 14, data pathways (e.g., buses) 16 and input/output devices 18 connecting the processor, memory and input/output devices. The computer system 10 may be in communication with a plurality of medical clinical databases using any suitable arrangement and any suitable devices such as the Internet, however, any suitable network might be used. The medical databases comprise a plurality of patient data. It is not necessary that the patient data from the clinical databases be obtained from a network. For example, the patient data can be made available from a local database coupled to the medical decision support system.

The processor 12 accepts instructions and data from the memory 14 and performs various data processing functions of the medical decision support system like data acquisition, data mining and medical prognosis. The processor 12 includes an arithmetic logic unit (ALU) that performs arithmetic and logical operations and a control unit that extracts instructions from memory 14 and decodes and executes them, calling on the ALU when necessary.

The memory 14 stores a variety of data computed by the various data processing functions of the medical decision support system. The data comprises biochemical laboratory tests, genetic information, radiological images and scans, environmental risk factors, longitudinal medical data related to patients, information on physical exams such as height, weight, blood pressure, physician actions and recommendations for a patient, expert opinion rule bases, serial imaging information etc. Serial imaging information includes radiological images collected over time from devices such as a X-ray scanner, a magnetic resonance image scanner, a positron emission tomography (PET) device, etc.

The memory 14 generally includes a random-access memory (RAM) and a read-only memory (ROM); however, there may be other types of memory such as programmable read-only memory (PROM), erasable programmable read-only memory (EPROM) and electrically erasable programmable read-only memory (EEPROM). Also, the memory 14 preferably contains an operating system, which executes on the processor 12. The operating system performs basic tasks that include recognizing input, sending output to output devices, keeping track of files and directories and controlling various peripheral devices. The information in the memory 14 might be conveyed to a human user through the input/output devices, and data pathways (e.g., buses) 16, in some other suitable manner.

The input/output devices may comprise a keyboard 20 and a mouse 22 that enter data and instructions into the computer system 10. Also, a display 24 may be used to allow a user to see what the computer has accomplished. Other output devices may include a printer, plotter, synthesizer and speakers. A communication device 26 such as a telephone or cable modem or a network card such as an Ethernet adapter, local area network (LAN) adapter, integrated services digital network (ISDN) adapter, or Digital Subscriber Line (DSL) adapter, enables the computer system 10 to access other computers and resources on a network such as a LAN or a wide area network (WAN). A mass storage device 28 may be used to allow the computer system 10 to permanently retain large amounts of data. The mass storage device 28 may include all types of disk drives such as floppy disks, hard disks and optical disks, as well as tape drives that can read and write data onto a tape that could include digital audio tapes (DAT), digital linear tapes (DLT), or other magnetically coded media.

The above-described computer system 10 can take the form of a hand-held digital computer, personal digital assistant computer, notebook computer, personal computer, workstation, mini-computer, mainframe computer or supercomputer.

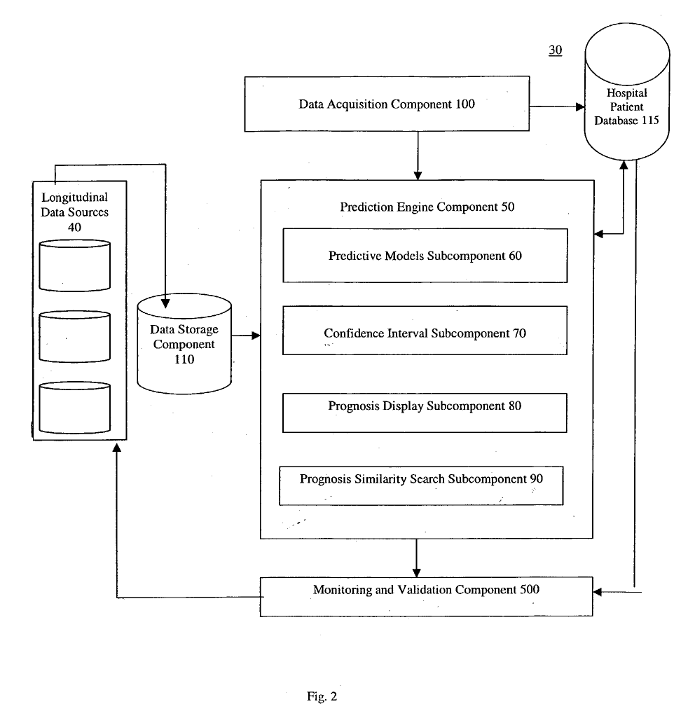

FIG. 2 shows a top-level component architecture diagram of a medical decision support system 30 that operates on the computer system 10 of FIG. 1. The decision support system 30 comprises longitudinal data sources 40, a data storage component 110, a hospital patient database 115, a data acquisition component 100, a prediction engine component 50 and a monitoring and validation component 500. The data storage component 110 comprises information of patients with various diseases. This information includes longitudinal medical data compiled from the longitudinal data sources 40, information on physical exams such as height, weight, blood pressure, outcome scales measuring symptom severity, medications, co-morbidity, serial imaging information, baseline information such as genetic test data, socio-demographic characteristics, family history, disease or symptom history, physician actions or recommendations for the patient and expert opinion rule bases. One of ordinary skill in the art will recognize that the above listing of longitudinal medical data is for illustrative purposes and is not meant to limit the other types of data that the medical decision support system 30 can use.

The hospital patient database 115 is coupled to the data acquisition component 100 and the prediction engine component 50. The hospital patient database 115 contains medical information relevant to patient visits. The hospital patient database 115 comprises information collected from a single hospital or a Health Maintenance Organization (HMO), or a combined organization of hospitals with centralized data warehousing. The hospital patient database 115 is used to store information collected from patient visits. The information includes demographics, patient physician communications, medical reports, imaging scan information, laboratory reports, physician recommendations etc.

The data acquisition component 100 acquires new patient data for patient diagnosis and prognosis. The data acquisition component 100 allows clinicians to acquire medical information based on specific patient requirements.

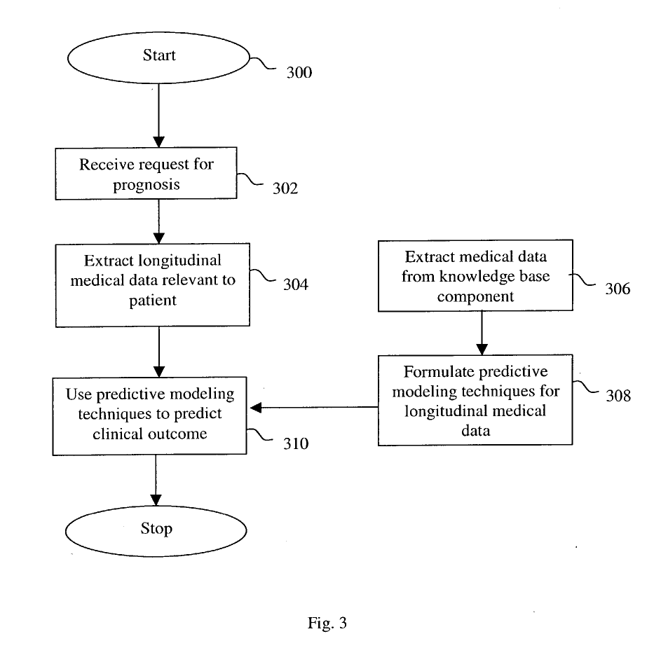

The prediction engine component 50 is configured to predict a clinical outcome for a patient. The prediction engine component 50 comprises a predictive models subcomponent 60, a confidence interval subcomponent 70, a prognosis display subcomponent 80 and a prognosis similarity search subcomponent 90. The predictive models subcomponent 60 comprises a plurality of predictive modeling techniques to predict a clinical outcome for a patient. The clinical outcome may indicate outcomes in the patient's condition such as disease status, disease severity, disease progression, risk of hospitalization, risk of mortality, and time to events such as survival or hospitalization. The predictive modeling techniques are formulated using specialized data mining techniques described below. These data mining techniques have been made to take into consideration correlations in repeated measurements associated with the longitudinal medical data to predict the clinical outcome for the patient. The longitudinal medical data corresponds to multiple clinical patient visits spread over time. FIG. 3 describes the process of prediction of the clinical outcome in further detail. The predictive models subcomponent 60 also categorizes a patient based on a degree of risk associated with the predicted clinical outcome. The degree of risk corresponds to a rate of decline in the patient's condition based on the predicted clinical outcome. The categorization identifies the patient as a high-risk, medium-risk or a low-risk patient.

The confidence interval subcomponent 70 computes a confidence interval for the predicted clinical outcome for a required measure of confidence in the prediction. In one embodiment of the invention, the confidence interval used is a 95% confidence interval. The confidence measure associated with the confidence interval is a statistical representation of the degree of accuracy associated with the predicted clinical outcome. The confidence interval denotes an upper and lower bound that the actual clinical outcome is likely to lie within, based on the value of the predicted clinical outcome. The confidence interval is derived based on an estimate of the variance of a set of parameters defined by the predictive modeling technique. The confidence interval measures the maximum variation that can be expected in the future clinical outcome for a given set of prognostic features.

The prognosis display subcomponent 80 displays a plurality of outputs related to the clinical outcome. In addition, the prognosis display subcomponent 80 tracks and analyzes a possible future course of the clinical outcome. The prognosis display subcomponent 80 also displays the time of occurrence of the clinical outcome. The prognosis display subcomponent also displays the deviation of the actual clinical outcome to the predicted clinical outcome, with respect to the computed confidence measure. Further, the prognosis display subcomponent 80 provides assistance in healthcare decision-making and medical treatment planning. FIG. 14 shows an example of a screen display that the prognosis display subcomponent 80 may present to a user of the medical decision support system 30.

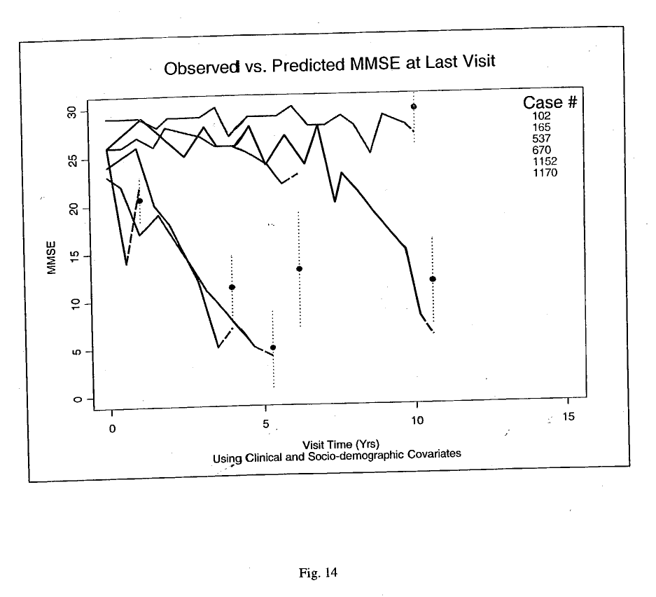

The prognosis similarity search subcomponent 90 generates a set of clinical recommendations for patients exhibiting a similar prognosis. The prognosis similarity search subcomponent 90 also displays a subset of patients with a similar prognosis. Patients with a similar prognosis are identified based on similar prognostic features and similar clinical outcomes. FIG. 13 is a flowchart describing the steps performed by the similarity search subcomponent 90 of the prediction engine component 50 of the medical decision support system 30.

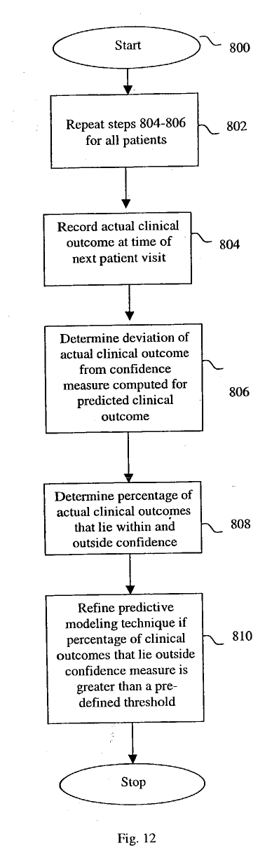

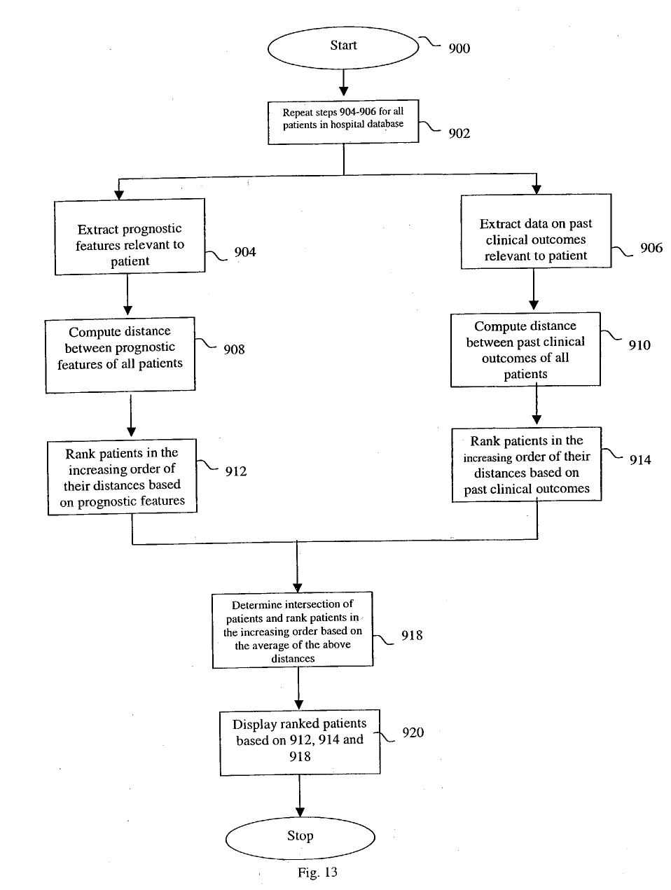

The monitoring and validation component 500 continuously monitors the system 30 and validates the predictive models being used to determine the prognosis for the patient. The monitoring and validation component 500 determines the deviation of the actual clinical outcome from the predicted clinical outcome for a patient. The monitoring and validation component 500 also reports error statistics associated with the predicted clinical outcome. FIG. 12 is a flowchart describing the steps performed by the monitoring and validation component 500 of the medical decision support system 30.

The data storage component 110 provides the data that is used to generate and validate the data mining techniques. These techniques are then applied to a patient of interest and a clinical outcome is predicted for the patient.

Below are further details of the knowledge base component 110, hospital patient database 115, data acquisition component 100, prediction engine component 50 monitoring and validation component 500 and the prognostic similarity search subcomponent 90 and their respective operation within the medical decision support system 30. In particular, FIGS. 3-11 describe further details of the predictive models subcomponent 60; FIG. 12 describes further details of the monitoring and validation component 500; FIG. 13 describes further details of the prognosis similarity search subcomponent 90; and FIG. 14 shows a screen display of a prognosis display subcomponent that is presented to a user of the medical decision support system 30.

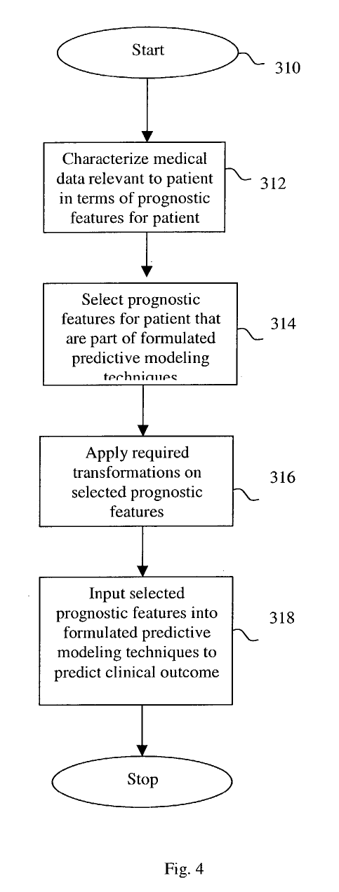

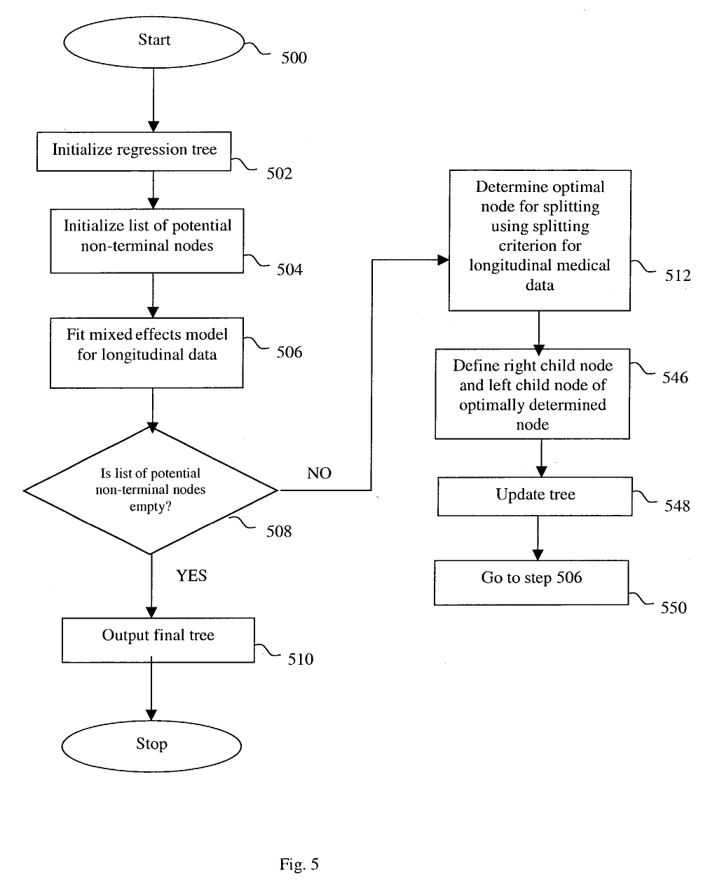

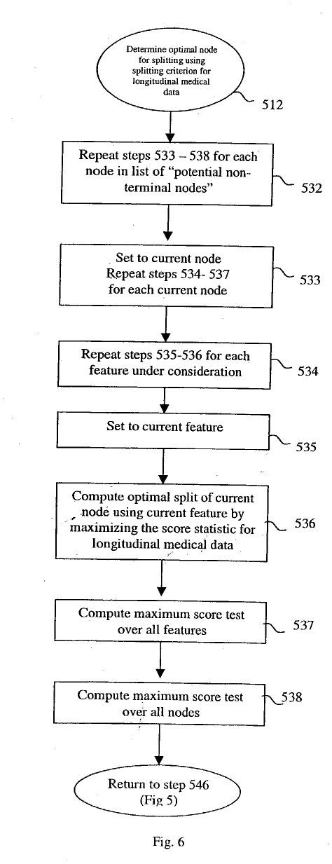

FIG. 3 is a high level flowchart describing the steps performed by the predictive models subcomponent 60 of the prediction engine component 50 of the medical decision support system 30. As shown, the process of FIG. 3 starts in step 300 and then passes to step 302. In step 302, a request for patient prognosis is received. The step involves activating the data acquisition component 100 to search for a plurality of longitudinal medical data related to the patient for patient prognosis. In step 304, the longitudinal medical data related to the patient is extracted. In step 310, the predictive modeling techniques are used to predict the clinical outcome for the patient. The step 310 is described in further detail in FIG. 4. The predictive modeling techniques are formulated using data mining techniques in step 308. The formulation includes automatic selection of prognostic features using the data mining techniques, determining a predictive model in terms of a function of the prognostic features of the patient, and estimation of a set of weights corresponding to the prognostic features. The selected prognostic features and the corresponding estimated weights are referred to as the model parameters. Step 308 extracts medical data relevant to various diseases from 306 to formulate the data mining techniques. The application of these techniques in the prediction of the clinical outcome are described in further details with reference to FIG. 5-FIG. 11.

FIG. 4 is a flowchart describing the “use predictive modeling techniques to predict the clinical outcome” step 310 of FIG. 3 in further detail. The process starts in step 310 and then passes to step 312. In step 312, medical data relevant to the patient is characterized in terms of prognostic features. Prognostic features relevant to the patient are measured and recorded for every patient visit and used by the predictive model to predict the clinical outcome. These repeated measurements constitute longitudinal medical data. Thus, the predictive model outputs for each patient the likely future course of the predicted outcome based on the patient's relevant prognostic features. In step 314, the set of prognostic features of the patient that correspond to the prognostic features defined by the predictive model are identified. For example, if age is not a prognostic feature of the predictive model, then this prognostic feature is not considered in the prediction of the clinical outcome for the patient. In step 316, the selected prognostic features are transformed. These transformations include, for example, data conversions suitable to a format used by the predictive model for prediction. In step 318, the selected prognostic features of the patient are input into the predictive model to predict the clinical outcome for the patient.

FIG. 5-FIG. 11 describe the application of data mining techniques for longitudinal medical data to predict the clinical outcome for a patient according to one embodiment of the invention. The data mining techniques deployed in the invention are preferably regression tree and neural network techniques. In one embodiment of the invention, these techniques are referred to as mixed effects regression trees for longitudinal medical data and mixed effects neural network techniques for longitudinal medical data respectively. The term mixed effects signifies a set of fixed effects and a set of random effects. The fixed effects correspond to the average effect of each prognostic feature on a set or population of patients with a given medical disorder. The random effects are patient specific and are measured over and above the fixed effects. They correspond to the effect of the prognostic feature on every individual patient.

FIG. 5 is a flowchart describing a data mining technique for longitudinal medical data based on regression trees. Regression trees are built through a process known as binary recursive partitioning which is an iterative process of splitting a given data set into partitions based on a set of rules, and then splitting the data set further on each of the branches of the tree. The data comprises pre-classified records that are used to determine the structure of the tree. The tree structure is defined in terms of a collection of nested rules based on a set of prognostic features.

The following steps describe the regression tree technique for longitudinal medical data. The technique incorporates correlations in repeated measurements associated with the longitudinal medical data related to the patient in the prediction of the clinical outcome. As shown, the process of FIG. 5 starts in step 500 and then passes to step 502. In step 502, the regression tree is initialized with the root node as the current tree. In step 504, the list of potential non-terminal nodes is generated and initialized with the root node. In step 506, a linear mixed effects model that is a standard modeling technique for longitudinal medical data is defined. The step takes in as input longitudinal medical data related to the patient. The mixed effects model for longitudinal medical data used in this step of the regression tree technique for longitudinal medical data is defined as follows:

yᵢ=Xᵢβ+Zᵢbᵢ+εᵢ, i=1, 2, . . . , n, (1)

wherein, yᵢ=(y_{i,1}, y_{i,2}, . . . , y_{i,ni}) are the nᵢ observations for the i^{th} patient, β is a p×1 vector of unknown fixed effects and Xᵢ is the nᵢ×p design matrix corresponding to the fixed effects for the i^{th} patient as defined by the current structure of the tree, bᵢ is a q×1 vector of unobservable random effects and Zᵢ is the nᵢ×q design matrix corresponding to the random effects, and εᵢ is the nᵢ×1 vector of random errors. In step 508, a check is made to determine if the list of potential non-terminal nodes is empty. If the condition in step 508 is true, the process passes to step 510. In step 510, the final regression tree representing the predicted clinical outcome for the patient is output and the process ends else, the process passes to step 512. In step 512, the optimal node for splitting the regression tree is selected from the list of potential non-terminal nodes using splitting criteria for longitudinal medical data. Initially the node selected is the current node. Step 512 is explained in further detail in FIG. 6. In step 546, the right child node and left child node of the optimally determined node are defined. In step 548, the decision tree is updated. In this step, the right child node and the left child node are added to the list of leaf nodes of the regression tree and the current node is removed from the list of potential non-terminal nodes of the regression tree. Then the process loops back to step 506 in step 550.

FIG. 6 is a flowchart describing the “determine optimal node for splitting using the splitting criterion for longitudinal medical data” step 512 of FIG. 5 in further detail. A split of a node is defined as a partition based on a selected prognostic feature X of the patient. The selection of an optimal split involves the selection of the optimal feature among p possible features, on which the split will be defined and selection of the optimal split point. Thus, if the k^{th} feature is selected optimally, and the optimal split point for this feature is γₖ, then the optimal splitting criterion at the node is defined as follows:

Left Child Node: k^{th} feature≦γₖ; Right Child Node: k^{th} feature>γₖ.

The optimal split point γₖ is determined by using a maximized score test determined from equation (1), which is known to skilled artisans. The node that gives the maximum value of the score test is selected as the node for splitting. The process starts in step 512 and passes to step 532. Step 532, repeats steps 533-538 for each node in the list of potential non-terminal nodes. In step 533, the first node in the list of potential non-terminal nodes is initialized to the current node. Step 533 repeats steps 534-537 for each current node. Then the process passes to step 534. Step 534 repeats steps 535-536 for each prognostic feature X of the patient under consideration. In step 535, the first feature is initialized to the current feature. In step 536, the optimal split of the current node using the current feature is computed by maximizing the score statistic. The score statistic is modified to take into consideration the longitudinal medical data. The optimal feature for splitting is chosen by maximizing the score test among all possible feature variables. In step 537, the maximum score test over all the prognostic features are computed. In step 538, the maximum score over all nodes is computed. Then the process passes back to step 546 of FIG. 5.

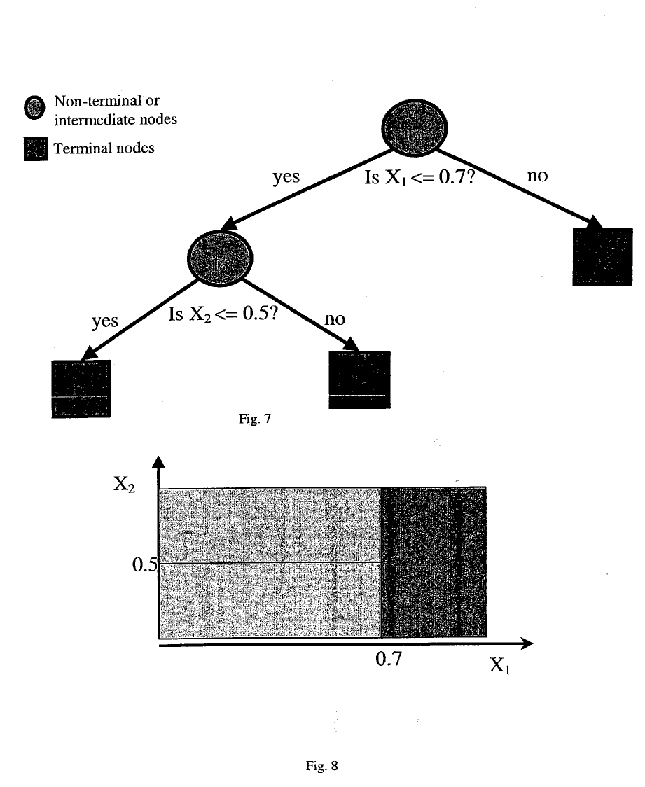

FIG. 7 shows the structure of a regression tree growing with two prognostic features X₁ and X₂. As explained above, regression trees are built through a process known as binary recursive partitioning. The regression tree technique involves repeated subdivisions of a data set on the basis of the choice of an optimal binary recursive partitioning of the feature space. The partitioning of the feature space by a sequence of binary splits defines a set of terminal nodes.

FIG. 8 shows the partition of the feature space corresponding to the splits defined by the regression tree shown in FIG. 7.

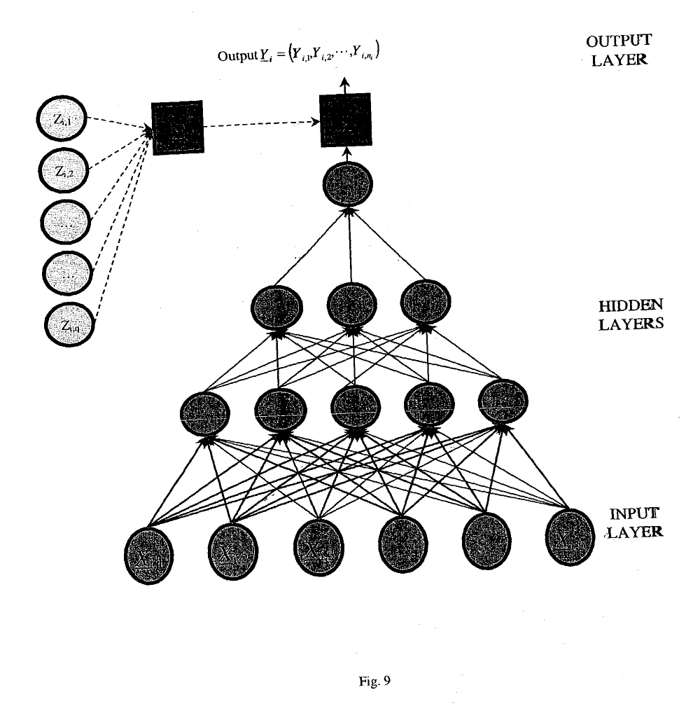

FIG. 9 is an illustration of a data mining technique for longitudinal medical data based on neural networks. The neural network technique for longitudinal medical data comprises a vector of observations on a prognostic feature X corresponding to the repeated measurements pertaining to the longitudinal medical data. In contrast, an input signal into a node of the input layer of a standard neural network is a single observation on a given prognostic feature X. Thus, for a given patient i, the standard neural network takes as input, a single value for a prognostic feature Xₖ and this feature receives a weight βₖ. In case of the mixed effects neural network, the input from a prognostic feature Xₖ is the nᵢ×1 vector Xᵢₖ=(X_{ik,1}, X_{ik,2}, . . . , X_{ik,n}_{ᵢ}) Therefore, every element of the vector receives a weight βₖ. This is different from treating each element of the nᵢ×1 vector as different observations in a standard neural network. The output variable in a standard neural network represents a one-dimensional measurement for each observation. In case of a mixed effects neural network, this is a nᵢ×1 vector Yᵢ=(Y_{i,1},Y_{i,2}, . . . , Y_{i,n}_{ᵢ}). The random effects for each subject consists of a nᵢ×1 vector arising out of the q random effects parameters and nᵢ×q dimensional design matrix corresponding to the random effects, Zᵢ. The random effects are added to the neural network to give the final output.

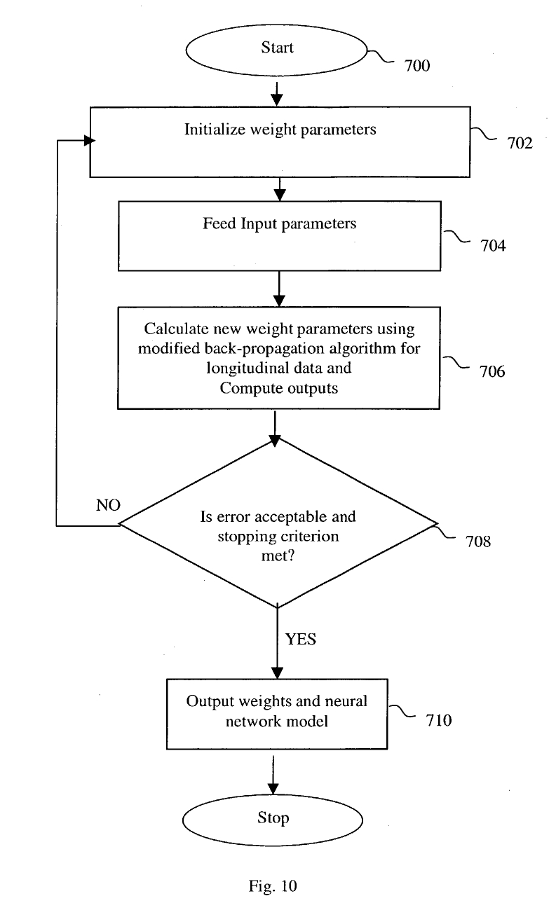

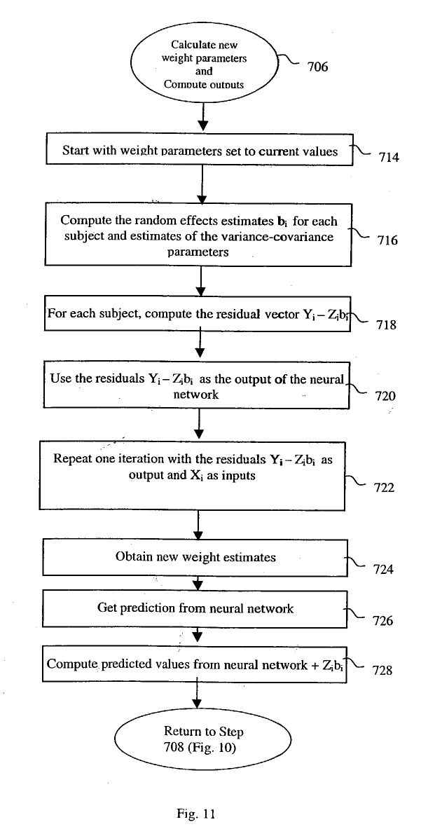

FIG. 10 is a flowchart describing the data mining technique for longitudinal medical data based on neural networks. The mixed effects neural networks technique for longitudinal medical data is modeled as:

yᵢ=f(Xᵢ,β)+Zᵢbᵢ+εᵢ, wherein (2)

f represents the neural network with inputs Xᵢ and weight parameters β. Here, yᵢ=(y_{i,1}, y_{i,2}, . . . , y_{i,ni}) are the nᵢ observations for the i^{th} patient, β is a p×1 vector of unknown fixed effects and Xᵢ is the nᵢ×p design matrix corresponding to the fixed effects, bᵢ is a q×1 vector of unobservable random effects and Zᵢ is the nᵢ×q design matrix corresponding to the random effects, and εi is the nᵢ×1 vector of random errors.

The process starts in step 700 and passes to step 702. In step 702, the weight parameters β are initialized. In step 704, the input parameters are input into the neural network. These parameters include the fixed effects Xᵢ and random effects, Zᵢ. In step 706, the new weight parameters are calculated and the outputs are computed. This step uses the standard back propagation algorithm of the neural networks technique to calculate the weight parameters and compute the outputs. In the invention, the standard back propagation algorithm has been modified to take into account the effects of the longitudinal medical data. Step 706 is explained in further detail in FIG. 11. In step 708, a check is made to determine if the random errors are acceptable and if the stopping criterion is met. The stopping criterion is based on a marginal likelihood criterion modified for longitudinal data that simultaneously minimizes of the fixed effects weights β and the random effects parameters, bᵢ's. If the condition of step 708 is true, the process passes to step 710 else the process loops back to step 702. In step 710, the weights are output along with the neural network comprising the predicted outcome for the patient.

FIG. 11 describes the “calculate new weight parameters and compute outputs” step 706 of FIG. 10 in further detail. The following steps describe the standard back propagation algorithm for neural networks modified to take into account the effects of the longitudinal medical data in the prediction of the clinical outcome for the patient. In step 714, the weight parameters are initialized to current values. In step 716, the random effects estimates bᵢ for each subject and estimates of the variance-covariance parameters are computed. In step 718, a residual vector Yᵢ-Zᵢbᵢ for each patient, is computed. In step 720, the residuals Yᵢ-Zᵢbᵢ are used as the output of the neural network. In step 722, one iteration of the back propagation algorithm for neural networks with the residuals Yᵢ-Zᵢbᵢ as output and Xᵢ as inputs is performed. In step 724, a set of new weight estimates is obtained. In step 726, a prediction is obtained from the neural network. In step 728, the predicted values form the neural network and Zᵢbᵢ are computed and output. Then the process passes back to step 708 of FIG. 10.

FIG. 12 is a flowchart describing the steps performed by the monitoring and validation component 500 of the medical decision support system 30. As shown, the process starts in step 800 and then passes to step 802. Step 802 repeats steps 804-806 for all patients in the hospital patient database 115. In step 804, the actual clinical outcome at the time of the next patient visit is recorded. In step 806, the deviation of the actual clinical outcome from the predicted clinical outcome is determined. In step 808, the percentage of actual clinical outcomes that lie within and outside the confidence measure are determined. In step 810, the monitoring and validation component 500 refines the predictive modeling techniques if the percentage of clinical outcomes that lie outside the confidence measure is greater than a pre-defined threshold.

FIG. 13 describes the steps performed by the prognosis similarity search subcomponent 90 of the prediction engine component 50 of the medical decision support system 30. As shown, the process starts in step 900 and then passes to step 902. Step 902 repeats steps 904-906 for all patients in the hospital patient database 115. In step 904, the prognostic features relevant to the patient are extracted. In step 906, the data on past clinical outcomes relevant to the patient is extracted. In step 908, a distance measure is computed between the prognostic features of all patients. In step 910, a distance measure between past clinical outcomes of all patients is computed. The distance measure is a degree of similarity of patients based on prognostic features and past clinical outcomes. In step 912, the patients are ranked in the increasing order of their distances based on prognostic features. These patients are similar only based on prognostic features. In step 914, the patients are ranked in the increasing order of their distances based on past clinical outcomes. These patients are similar only based on past clinical outcomes. In step 918, an intersection of patients from steps 912 and 914 is determined and the patients are ranked in the increasing order of the average of the distances based on the computed distance measures. Patients belonging to the intersection set are similar with respect to both prognostic features and past clinical outcomes. In step 920, the ranked patients are displayed to the physician. The physician can make use of the rankings obtained from either of the steps, 912, 914 or 918 to provide patient actions and recommendations.

FIG. 14 shows a screen display that the prognosis display subcomponent 80 may present to a user of the medical decision support system 30 as it operates in the manner described with reference to FIGS. 3-13. FIG. 14 displays the predicted outcome in terms of the MMSE value. The MMSE refers to the Mini-Mental Status Examination as applicable to a nuerodegenerative disorder such as Alzheimer's disease. Clinical outcomes in Alzheimer's disease are generally measured using the MMSE value. One of ordinary skill in the art will recognize that the above display is for illustrative purposes and is not meant to limit the other types of disorders that the medical decision support system 30 can determine a prognosis for. The X-axis represents patient visits in years and the Y-axis represents the MMSE value. The dotted lines in the display represent the confidence measure of the predicted clinical outcome. The case# represents the future course of the clinical outcome for various patients. The covariates refer to the prognostic features.

The foregoing flow charts, block diagrams and screen shots of this disclosure show the functionality and operation of the medical decision support system 30. In this regard, each block/component represents a module, segment, or portion of code, which comprises one or more executable instructions for implementing the specified logical function(s). It should also be noted that in some alternative implementations, the functions noted in the blocks may occur out of the order noted in the figures or, for example, may in fact be executed substantially concurrently or in the reverse order, depending upon the functionality involved. Also, one of ordinary skill in the art will recognize that additional blocks may be added. Furthermore, the functions can be implemented in programming languages such as C++ or JAVA; however, other languages can be used such as Perl, Javascript and Visual Basic

The various embodiments described above comprise an ordered listing of executable instructions for implementing logical functions. The ordered listing can be embodied in any computer-readable medium for use by or in connection with a computer-based system that can retrieve the instructions and execute them. In the context of this application, the computer-readable medium can be any means that can contain, store, communicate, propagate, transmit or transport the instructions. The computer readable medium can be an electronic, a magnetic, an optical, an electromagnetic, or an infrared system, apparatus, or device. An illustrative, but non-exhaustive list of computer-readable mediums can include an electrical connection (electronic) having one or more wires, a portable computer diskette (magnetic), a random access memory (RAM) (magnetic), a read-only memory (ROM) (magnetic), an erasable programmable read-only memory (EPROM or Flash memory) (magnetic), an optical fiber (optical), and a portable compact disc read-only memory (CDROM) (optical).

Note that the computer readable medium may comprise paper or another suitable medium upon which the instructions are printed. For instance, the instructions can be electronically captured via optical scanning of the paper or other medium, then compiled, interpreted or otherwise processed in a suitable manner if necessary, and then stored in a computer memory.

It is apparent that there has been provided, a method, system and computer product for prognosis of a medical disorder. While the invention has been particularly shown and described in conjunction with a preferred embodiment thereof, it will be appreciated that variations and modifications can be effected by a person of ordinary skill in the art without departing from the scope of the invention.

1. A method for determining a medical prognosis of a patient with a medical disorder, comprising:
receiving a request to provide prognostic decision support for the patient;
extracting a plurality of medical data relevant to the patient, wherein the plurality of medical data comprises a plurality of longitudinal medical data; and
using a plurality of predictive modeling techniques to predict at least one clinical outcome for the patient from the plurality of medical data, wherein the plurality of predictive modeling techniques are formulated using a plurality of data mining techniques that are capable of detecting correlations in repeated measurements associated with the plurality of longitudinal medical data and wherein the plurality of predictive modeling techniques utilize the correlations for determining the medical prognosis for the patient.

2. The method of claim 1, wherein the medical disorder comprises at least one of neurodegenerative disorders, cardiovascular disorders and cancer.

3. The method of claim 1, wherein the predictive modeling techniques comprise using a regression tree for longitudinal medical data with a plurality of rules to predict the clinical outcome.

4. The method of claim 1, wherein the predictive modeling techniques comprise using a neural network technique for longitudinal medical data that models a relationship between the plurality of longitudinal medical data and the clinical outcome.

5. The method of claim 1, further comprises categorizing the patient based on a degree of risk associated with the predicted clinical outcome, wherein the categorization identifies the patient as a high-risk, medium-risk or a low-risk patient.

6. The method of claim 5, wherein the degree of risk corresponds to a rate of decline in the patient's condition based on the predicted clinical outcome.

7. The method of claim 1, further comprises displaying a plurality of outputs related to the predicted clinical outcome for the patient.

8. The method of claim 7, further comprising tracking and analyzing a trend in the predicted clinical outcome, wherein the trend represents a possible future course of the predicted clinical outcome.

9. The method of claim 8, wherein the tracking further comprises assisting in healthcare decision making and medical treatment planning.

10. The method of claim 7, wherein the plurality of outputs comprise displaying a time of occurrence of the predicted clinical outcome.

11. The method of claim 7, wherein the plurality of outputs comprise displaying a confidence measure for the predicted clinical outcome, wherein the confidence measure represents a degree of accuracy associated with the predicted clinical outcome.

12. The method of claim 1, further comprises validating the predicted clinical outcome over time.

13. The method of claim 1, further comprises acquiring new patient data for patient prognosis.

14. The method of claim 1, further comprises generating a plurality of clinical recommendations for a plurality of patients by identifying and comparing patients exhibiting similar prognosis.

15. A medical decision support system for prognosis of a medical disorder, comprising:
a data storage component configured to store a plurality of medical patient data comprising longitudinal medical data; and
a prediction engine component coupled to the data storage component comprising a plurality of predictive modeling tools that predict at least one clinical outcome for a patient, wherein the plurality of predictive modeling tools are formulated using a plurality of data mining tools that are capable of detecting correlations in repeated measurements associated with the plurality of longitudinal medical data and wherein the plurality of predictive modeling tools utilize the correlations for determining the medical prognosis for the patient

16. The system of claim 15, wherein the medical disorder comprises at least one of neurodegenerative disorders, AIDS, cardiovascular disorders and cancer.

17. The system of claim 15, wherein the plurality of predictive modeling tools comprise a regression tree technique for longitudinal medical data that uses a plurality of rules to predict the clinical outcome.

18. The system of claim 15, wherein the plurality of predictive modeling tools comprise a neural network technique for longitudinal medical data that models a relationship between the plurality of longitudinal medical data and the clinical outcome.

19. The system of claim 15, wherein the prediction engine component is further configured to categorize a patient based on a degree of risk associated with the predicted clinical outcome, wherein the categorization identifies the patient as a high-risk, a medium-risk or a low-risk patient.

20. The system of claim 19, wherein the degree of risk corresponds to a rate of decline in the patient's condition based on the predicted clinical outcome.

21. The system of claim 15, wherein the prediction engine component comprises a confidence interval subcomponent configured to compute a confidence measure for the predicted clinical outcome that represents a degree of accuracy associated with the predicted clinical outcome.

22. The system of claim 15, wherein the prediction engine component comprises a prognosis display subcomponent configured to track and analyze a trend in the clinical outcome, wherein the trend represents a possible future course of the predicted clinical outcome.

23. The system of claim 22, wherein the prognosis display subcomponent is further configured to display the time of occurrence of the predicted clinical outcome.

24. The system of claim 22, wherein the prognosis display subcomponent is further configured to display a confidence measure associated with the predicted clinical outcome.

25. The system of claim 22, wherein the prognosis display subcomponent is further configured to provide assistance in healthcare decision making and medical treatment planning.

26. The system of claim 15, wherein the prediction engine component comprises a prognosis similarity search subcomponent configured to generate a plurality of clinical recommendations for a plurality of patients exhibiting similar prognosis.

27. The system of claim 15, further comprises a monitoring and validation component coupled to the prediction engine component and configured to monitor and validate the predicted clinical outcome over time.

28. The system of claim 15, further comprises a data acquisition component coupled to the prediction engine component and configured to acquire new patient data for patient prognosis.

29. A computer-readable medium storing computer instructions for instructing a computer system to determine a medical prognosis of a patient with a medical disorder, the computer instructions comprising:
receiving a request to provide prognostic decision support for the patient;
extracting a plurality of medical data relevant to the patient; wherein the plurality of medical data comprises a plurality of longitudinal medical data; and
using a plurality of predictive modeling techniques to predict at least one clinical outcome for the patient from the plurality of medical data, wherein the plurality of predictive modeling techniques are formulated using a plurality of data mining techniques that are capable of detecting correlations in repeated measurements associated with the plurality of longitudinal medical data and wherein the plurality of predictive modeling techniques utilize the correlations for determining the medical prognosis for the patient

30. The computer-readable medium of claim 29, wherein the medical disorder comprises at least one of neurodegenerative disorders, AIDS, cardiovascular disorders and cancer.

31. The computer-readable medium of claim 29, wherein the predictive modeling techniques comprise processing instructions for using a regression tree for longitudinal medical data with a plurality of rules to predict the clinical outcome.

32. The computer-readable medium of claim 29, wherein the predictive modeling techniques comprise processing instructions for using a neural network technique for longitudinal medical data that models a relationship between the plurality of longitudinal medical data and the clinical outcome.

33. The computer-readable medium of claim 29, further comprises instructions for categorizing the patient based on a degree of risk associated with the predicted clinical outcome, wherein the categorization identifies the patient as a high-risk, medium-risk or a low-risk patient.

34. The computer-readable medium of claim 33, wherein the degree of risk corresponds to a rate of decline in the patient's condition based on the predicted clinical outcome.

35. The computer-readable medium of claim 29, further comprises instructions for displaying a plurality of outputs related to the predicted clinical outcome for the patient.

36. The computer-readable medium of claim 35, further comprising instructions for tracking and analyzing a trend in the predicted clinical outcome, wherein the trend represents a possible future course of the predicted clinical outcome.

37. The computer-readable medium of claim 36, wherein the tracking further comprises instructions for assisting in healthcare decision making and medical treatment planning.

38. The computer-readable medium of claim 35, wherein the plurality of outputs comprise instructions for displaying a time of occurrence of the predicted clinical outcome.

39. The computer-readable medium of claim 35, wherein the plurality of outputs comprise instructions for displaying a confidence measure for the predicted clinical outcome, wherein the confidence measure represents a degree of accuracy associated with the predicted clinical outcome.

40. The computer-readable medium of claim 29, further comprises instructions for validating the predicted clinical outcome over time.

41 The computer-readable medium of claim 29, further comprises instructions for acquiring new patient data for patient prognosis.

42. The computer-readable medium of claim 29, further comprises instructions for generating a plurality of clinical recommendations for a plurality of patients by identifying and comparing patients exhibiting similar prognosis.

43. A method for determining a medical prognosis of a patient with a medical disorder, comprising:
extracting a plurality of medical data relevant to the patient, wherein the plurality of medical data comprises a plurality of longitudinal medical data;
using a plurality of predictive modeling techniques to predict at least one clinical outcome for the patient from the plurality of medical data, wherein the plurality of predictive modeling techniques are formulated using a plurality of data mining techniques that are capable of detecting correlations in repeated measurements associated with the plurality of longitudinal medical data and wherein the plurality of predictive modeling techniques utilize the correlations for determining the medical prognosis for the patient

44. The method of claim 43, wherein the predictive modeling techniques comprise using a regression tree for longitudinal medical data with a plurality of rules to predict the clinical outcome.

45. The method of claim 43, wherein the predictive modeling techniques comprise using a neural network technique for longitudinal medical data that models a relationship between the plurality of longitudinal medical data and the clinical outcome.

46. A computer-readable medium storing computer instructions for instructing a computer system to determine a medical prognosis of a patient with a medical disorder, comprising:
extracting a plurality of medical data relevant to the patient, wherein the plurality of medical data comprises a plurality of longitudinal medical data;
using a plurality of predictive modeling techniques to predict at least one clinical outcome for the patient from the plurality of medical data, wherein the plurality of predictive modeling techniques are formulated using a plurality of data mining techniques that are capable of detecting correlations in repeated measurements associated with the plurality of longitudinal medical data and wherein the plurality of predictive modeling techniques utilize the correlations for determining the medical prognosis for the patient.

47. The computer-readable medium of claim 46, wherein the predictive modeling techniques comprise processing instructions for using a regression tree for longitudinal medical data with a plurality of rules to predict the clinical outcome.

48. The method of claim 46, wherein the predictive modeling techniques comprise processing instructions for using a neural network technique for longitudinal medical data that models a relationship between the plurality of longitudinal medical data and the clinical outcome.
